(19) United States
(12) Patent Application Publication
(10) Pub. No.: US 2021/0228361 A1
(43) Pub. Date: Jul. 29, 2021

(54) A Bone Implant

(71) Applicants: Zoran Lee PETRIC, Sydney, New South Wales (AU); Martin SCHOLSEM, Kogarah, New South Wales (AU)

(72) Inventors: Zoran Lee PETRIC, Sydney, New South Wales (AU); Martin SCHOLSEM, Kogarah, New South Wales (AU)

(21) Appl. No.: 17/255,524

(22) PCT Filed: Jun. 28, 2019
(86) PCT No.: PCT/IB2019/055482

§ 371 Date: Dec. 23, 2020

(30) Foreign Application Priority Data
Jul. 1, 2018 (AU) 2018902383
Apr. 11, 2019 (AU) 2019901245

(51) Int. Cl. A61F 2/28; A61B 17/70; A61B 17/86

(52) U.S. Cl. CPC A61F 2/28; A61B 17/7032; A61B 2017/564; A61B 17/8685; A61B 17/864

(57) ABSTRACT

There is described a bone implant comprising at least one means for providing at least one bone stimulation dynamic interaction to at least one area of a bone-implant interface formed when the implant is inserted into bone, wherein the bone stimulation is one or more of bone growth, bone strengthening, bone densification and/or osseointegration between bone and the bone-implant interface.

### FIELD OF THE INVENTION

The present invention relates to bone growth promoting devices, and methods for promoting bone growth, particularly osseointegration bone growth at the contact point between bone and the surface of an implant, e.g., having an internally loaded mechanism for promoting osseointegration through a process herein termed ‘dynamic osseointegration’.

### BACKGROUND

Medical implants are devices or tissues that are placed inside or on the surface of the body. Medical implants include prosthetics, which are intended to replace missing body parts. Other implants interact with support tissues, including bone. Bone implants used in surgical techniques include dental implants, bone-anchored hearing aids, spinal fusion implants, and endo-exo prostheses.

Many bone implants have a screw thread as a bone engagement element, e.g., a bone screw. Typically, a bone screw has a turning head on one end of a cylindrical body with spiral or helical threads running along the outer surface of the body which convert torsional forces into compression and linear displacement when rotated. As the screw passes through bone, the thread grips surrounding bone into which it is being screwed.

In particular, orthopaedic screws are bone screws used for attachment of implants to bone and bone to bone fixation, and include compression screws, cannulated screws for minimally invasive surgery, dental screws, pedicle screws, etc. Screws can be designed for insertion into cancellous or cortical bone or for insertion into various parts of the skeleton. Pedicle screws are one example of a bone implant and pedicle screw fixation is a useful surgical technique that has been applied extensively for many years to various types of surgery of the spine, e.g, in orthopaedic surgery for spinal deformity and to treat trauma, tumour and degenerative cases where pedicle screws enhance fusion rates. Vertebral pedicles provide a suitable location for screw attachment due to the geometry of the vertebrae and the characteristic strength of the bone. In a patient with suitable bone density and strength, the pedicles may be used in instrumentation procedures to affix rods, cables, plates and other structures to the spine. In particular, the instrumentation is secured to the spinal column using pedicle screws which are inserted through the bony pedicle and into the anterior vertebral body. A pedicle screw provides means for gripping a spinal segment. The screw head for a pedicle screw is typically a tulip head which enables the connection of the pedicle screw to a rod used for spinal fusion/stabilisation.

Although the use of pedicle screws provides advantages, there are many reports of complications such as loosening, pull-out or breakage of screws. Pedicle screws are particularly known to fail at the end of long constructs, in patients with poor bone quality and when the forces applied to the construct are excessive. Loosened screws are associated with decreased pull-out strength and extraction torque due to poor purchase power in bone and can be regarded as a type of instrument failure.

Various methods have been used to improve implant and pedicle screw fixation including increasing the diameter of screws, use of specific coatings, augmentation technique using polymethylmethacrylate and most recently expandable pedicle screws. However, loosening of the implants/screws is still frequent and pathological studies have shown that loosening occurs when the implant-bone interface does not fuse with bone. Furthermore, where bone quality is poor, e.g., osteoporotic bone, or other defective bone formation with increased porosity and increased bone fragility, loosening or other implant/screw failures are more likely.

Dental implants are other category of bone implant where improved fixation is desirable, particularly in patients with poor bone quality. Dental implant surgery is a procedure that replaces tooth roots with metal, screwlike posts and replaces damaged or missing teeth with artificial teeth that look and function much like real ones. A secure implant for new teeth requires the bone to heal tightly around the implant. Because healing requires time, the process can take many months.

Two types of fixation are necessary for a successful bone implant installation. Mechanical friction or primary stability initially holds the implant in place. Primary or mechanical fixation of the implant to the recipient bone occurs via the friction action of the implant surfaces, for example, the thread, against the bone walls of the implant osteotomy. Mechanical fixation is the primary contributing factor to the initial stability of the implant itself. However, for long-term stabilisation, biological fixation or osseointegration of the inserted surfaces of the implant with the surrounding bone is necessary. Osseointegration, that is, direct skeletal integration, occurs where bone tissue surrounding the implant grows over time and essentially fuses with the surface of the implant.

Contact between bone and the implant surface can be observed microscopically. The principle factors for achieving direct fixation via osseointegration have been reported to include: implant biocompatibility, the implant surface properties; quality of the host bone; implant osteotomy quality and preparation; surgical site preparation; loading conditions; implant design; and preventing initial and chronic infections. Following implant insertion, during the healing process which takes place over a number of week or months, ideally the implant becomes fully integrated into the surrounding bone. The understanding in the art is that the optimal direct bone implant interface occurs when an implant is allowed to heal in bone undisturbed, e.g., for example unloaded. As explained, prior art implants often suffer from poor osseointegration and as a result premature failure when loaded.

It is an object of a preferred embodiment of the invention to provide improved implants which are capable of promoting osseointegration which in turn results in superior biomechanical fixation in faster timeframes and/or a superior bone-implant osseointegration than currently observed using conventional implants and surgical methods under otherwise equivalent conditions. It is an object of a preferred embodiment of the invention to provide a bone structure enhancement implant which through application of the dynamic bone growth forces described herein, stimulates or changes the internal bony structures of a vertebra from normal or weak or cancellous or osteoporotic bone into an improved or otherwise stronger bone. Suitably, the resultant stimulated stronger or enhanced bone would offer a better bone structure for the subsequent insertion of a load bearing implant such as a pedicle screw. It is an object of a preferred embodiment of the invention to provide a surgical method of implanting and/or loading a bone implant which leads to osseointegration which in turn results in superior biomechanical fixation in faster timeframes than currently observed using conventional implants and surgical methods under otherwise equivalent conditions.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### SUMMARY OF THE INVENTION

In a first aspect, the invention provides a bone implant comprising:

an implant body, an implant head, and optionally, an implant head extension for removeable attachment to the implant head, and

at least one mechanical means associated with the bone implant for providing at least one bone stimulation interaction to at least one area of a bone-implant interface formed when the implant is inserted into bone, wherein bone stimulation is one or more of bone growth, bone strengthening, bone densification and osseointegration between the bone-implant interface and/or the surrounding bone,

wherein the mechanical means comprises one or more actuatable elements arranged to provide active load stimulation to the bone-implant interface. The active load stimulation promotes bone growth, bone strengthening, and/or bone densification of the bone surrounding the bone-implant interface. By active load it is meant a dynamic load which is constantly changing in terms of level and/or frequency of applied force. Preferably, the bone stimulation interaction is a bone stimulation dynamic interaction. Suitably, the actuatable element(s) provide active load stimulation to the bone-implant interface.

Suitably, in one embodiment, one actuatable element is the implant head. In this case, desirably, attachment of the implant head to the implant body is via a moveable attachment wherein the implant head is translatable along a portion of a longitudinal axis of the implant and/or is rockable when positioned on the implant body. A moveable attachment of implant body and implant head allows for movement of the implant head relative to the implant body thereby causing micromovement of the implant body, at least until the head is replaced with a permanent head or the moveable head is locked down via fixation of a spinal rod for example to the implant head.

Preferably the implant head is fully removeable from the implant body.

Preferably, the mechanical means, e.g., the one or more of the actuatable elements, are freely mobile within one or more of the implant, implant body, implant head and/or the removable implant head extension such that on experiencing movement or other stimulation, the elements translate, for example, longitudinally, transversally, and/or radially within the implant body, implant head and/or the removable implant head extension.

The optional removable implant head extension is particularly suited to use of the implant in a two-stage surgical procedure as described herein, whereby the removable implant head extension is associated with the implant in a first surgical step to prevent tissue ingress into or around the head or internal parts of the implant body during the healing phase, and later during a second surgical step, the removable implant head extension is removed and replaced with a permanent implant head for completing the implant construct, for example a tulip head in the case of a pedicle screw implant. Alternatively, where one actuatable element of the mechanical means is a mobile implant head, e.g., in the pedicle screw case, when a surgical rod is placed and locked into the implant head during the second surgical step, this can cease the mobility of the mobile implant head thereby ending the dynamic stimulatory interaction.

Desirably, one or more actuatable elements comprise one or more discrete bodies that are freely mobile within the implant body, the implant head and/or the implant head extension. Suitably, one or more actuatable elements comprise grains, hollow or solid spheres, rolling or ball bearings, particles, and/or one or more translatable rods, pins, cylinders, or combinations thereof preferably a cannulated rod or cylinder.

Preferably, the bone implant further comprises at least one chamber, region, passageway, cannulation or orifice in the implant body, implant head and/or the implant head extension, comprising the mechanical means, preferably wherein the mechanical means is removable from the implant.

Suitably, the bone implant is dissolvable in situ. This is desirable for embodiments where bone strengthening and densification are required separate to or as a pre-treatment prior to spinal stabilisation or the like.

Preferably, the mechanical means moves with the bone implant on kinetic movement of a subject having the implant, or the mechanical means move independently of subject's kinetic movement, for example, controlled by a motor or other controlling device.

Suitably, the implant body, implant head and/or the implant extension head is cannulated.

Typically, the implant will include pores or other orifices into which osteoblasts and supporting connective tissue can migrate (bone infiltration).

The implant body facilitates insertion and retention of the implant into bone. Preferably, the implant head is engagable with a tool for insertion of the implant body into the bone, preferably a tulip head. Further, the implant can have a neck portion for connecting the implant body with an implant head.

When the implant is used in a two-step surgical procedure, it is important to facilitate access of an implant placed in the first surgical step after a set period of time allowed for healing, for example, a few weeks or months. Thus, the implants must be located accurately in their position deep under the skin and muscle layers. Also it must be possible to re-attach instruments to them: cannula in the case the clinician decides to use, e.g., bone cement to reinforce the implant, or in the case of a pedicle screw implant, fitting of a permanent head and/or insertion of a rod into the screw head. Therefore, to help achieve these goals, the implant head is preferably provided with a system preventing tissue or bone in growth such as a fine layer of organic polymer which is easily pierced by a surgical instrument but would prevent the tissue to adhering to the implant head. Further, the implant head is preferably visible to an external source. For example a preoperative spine CT and the software (spinal guidance) can guide the surgeon on the target like for a brain biopsy. Alternatively a robot, based on a preoperative or intraoperative CT, can cannulate the head of the implant independently. Desirably, the bone implant further comprises an implant body cover and/or an implant head cover, and/or an implant extension cover for closing off internal parts from tissue ingress. This is preferably a temporary cover which can be removed in a later procedure where access to the implant is required. Closing off internal parts from tissue ingress is an important consideration for two stage surgical use of the implant of the invention, as a permeant implant head, e.g., a tulip head can be fitted when sufficient bone growth, strengthen and/densification has occurred and the implant is ready for loading, e.g., with a spinal rod in the case of a pedicle screw, or if bone cement is to be inserted into the implant to strengthen the fixation in the bone. Where an implant head extension for removeable attachment to the implant head is included with the implant, the cover can be provided on the implant extension on a face located distally to the direction of implantation of the implant. Suitably, the cover is a temporary cover, such as, a pierceable cover formed from a layer of biocompatible material which can be penetrated by a K-wire or the like. Suitably materials include biocompatible materials including gelatin, silicon, collagen and combinations thereof, alone or with other known biocompatible membranes. Preferably, the removable implant head extension and/or the temporary cover has a shape and/or configuration that enables location under the subject's skin, for example, comprising a pointed, indented or textured surface for tactile location under the skin and/or a radio opaque material for location via CT or radiological methods, both manually and robotically.

Most preferably, the bone implant is a pedicle screw and further comprising an implant set or locking screw or cap for locking a spinal rod into the implant head. Desirably, the locking screw or cap for the pedicle screw comprises the mechanical means, preferably wherein the mechanical means is removable from the locking screw or cap.

In a second aspect, the invention provides a use of the bone implant of any one of the preceding claims, as a device for treating and/or preventing a bone wasting/weakening disease or disorder such as osteopenia and/or osteoporosis.

In a third aspect, the invention provides a bone implant of the first aspect, in a surgical technique, such as in the treatment, prevention and/or correction of one or more of spinal stenosis, spondylolisthesis, spinal deformities, fracture, pseudoarthosis, tumour resection, failed previous fusion, degenerative disc disease, dislocation, scoliosis, and kyphosis.

In a fourth aspect, the invention provides a kit of parts comprising a plurality of the implants of the first aspect.

In a fifth aspect, the invention provides a use of the bone implant of the first aspect in a method of promoting bone stimulation including bone growth, strengthening, densification and/or osseointegration.

In a sixth aspect, the invention provides a use of the bone implant of the first aspect in a two stage surgical method involving a first stage surgical step whereby a bone implant according to the first aspect is inserted into a bone at a first timepoint, followed by a second stage surgical step at a second timepoint in which a load is applied to the bone implant inserted in the first stage surgical step, whereby the interval between the first step and the second step is sufficient to allow an acceptable level of osseointegration to occur between the implant and bone, for example, at least 6 weeks.

Described herein is a bone implant comprising at least one means for providing at least one bone stimulation interaction to at least one area of a bone-implant interface formed when the implant is inserted into bone.

Suitably, the bone stimulation interaction is a bone stimulation dynamic interaction as described herein. Desirably, the bone stimulation is one or more of bone growth, bone strengthening, bone densification of the bone surrounding the implant, and/or, osseointegration between bone and the bone-implant interface.

Bone stimulation can be determined by in vivo or ex vivo methods. For example, in an ex vivo setting, for example, an animal study, bone stimulation can be determined by imaging (e.g., CT, micro CT, Atom Probe Tomography, etc.), mechanical testing (e.g., pull-out strength, resonance frequency analysis), scanning electron microscopy and histology, Raman spectroscopy. In in-vivo, for example, the human setting, bone stimulation can be determined by imaging-studies (e.g., x-rays, computerized tomography) looking for direct and indirect signs of healing. It tends to be more, challenging to evaluate osseointegration in the clinical setting as the implant cannot be retrieved for ex-vivo examination.

Suitably, the bone stimulation dynamic interaction is a bone growth, bone strengthening, and/or bone densification dynamic interaction provided to the bone surrounding the bone-implant interface.

Suitably, in one embodiment the bone stimulation dynamic interaction changes the internal bony structures of the bone, such as vertebral bone, from normal, weak, cancellous and/or osteoporotic bone into stronger bone. Such changes/stimulation can be identified by imaging studies, but also clinically, evidenced by a lower rate of screw loosening compared to the traditional implants. Bone stimulation can also be demonstrated by conducting an animal study.

Suitably, in one embodiment the bone stimulation dynamic interaction is an osseointegration-promoting dynamic interaction provided to at least one area of a bone-implant interface formed when the implant is inserted into bone. Suitably, the osseointegration promoting dynamic interaction provides an osseointegration-promoting force to the at least one area of a bone-implant interface.

There are several definitions for osseointegration. However, osseointegration is a direct contact between bone and an implant without interposed soft tissue layers. Osseointegration results in a direct bone anchorage to an implant body providing a secure foundation and/or support to a load bearing implant. A common description is that osseointegration represents a direct structural and functional connection between ordered living bone and the surface of a load carrying implant. Another description provides that osseointegration represents contact established without interposition of non-bone tissue between normal remodelled bone and an implant entailing a sustained transfer and distribution of load from implant to and within the bone tissue. Others have subdivided osseointegration into adaptive osseointegration corresponding to osseous tissue approximating the surface of the implant without apparent soft tissue interface at light microscopic level; and bio-integration which is a direct biochemical bone surface attachment confirmed at electron microscopic level. Others still defined it as a process whereby clinically asymptomatic rigid fixation of alloplastic materials is achieved and maintained, in bone during functional loading. Irrespective of the definition, osseointegration requires new bone formation around an implant and results from bone remodelling within bone tissue. Osseointegration is a direct contact between bone and an implant without interposed soft tissue layers.

Described herein is a bone implant comprising at least one means for providing at least one osseointegration-promoting dynamic interaction to at least one area of a bone-implant interface formed when the implant is inserted into bone. Described herein is a bone implant comprising at least one means for providing at least one bone densification dynamic interaction to at least one area of a bone-implant interface formed when the implant is inserted into bone.

It will be understood that a bone-implant interface is formed when the implant is inserted into a bone. The bone-implant interface represents a region of proximity between an area of bone tissue surrounding the implant and one or more surfaces of the implant. To ensure a good/snug fit and promote osseointegration, it is often the case during implantation that using a drill or a tap of smaller diameter than the screw diameter (e.g., around 1 mm smaller) such that implant insertion results in a best fit, snug fit within the bone

Suitably, the bone growth dynamic interaction and/or the osseointegration-promoting dynamic interaction is an active and changing interaction applied to the at least one area of a bone-implant interface. Desirably, the bone growth dynamic interaction and/or the osseointegration-promoting dynamic interaction is energy, for example, in the form of a force being applied to the bone-implant interface. Generation of a dynamic interaction at the bone-implant interface is thought to result in local stimulation of cellular responses associated with bone growth. The positive changes in cellular response accelerate healing and bone resorption and remodelling resulting in bone enhancement and/or faster and/or superior osseointegration at the bone-implant interface.

Herein the improved osseointegration process resulting from the application of the osseointegration-promoting dynamic interaction is designed ‘dynamic osseointegration’. Improvements in osseointegration at the bone-implant interface compared to classical implants can be verified by one or more osseointegration evaluation techniques as described herein. Suitable osseointegration of the implant can be determined by one or more of:
(a) clinical assessment where no instability (mobility), pain or neurological deficit during movement Is observed;
(b) exploratory surgical assessment;
(c) lack of movement at the level fused during the flexion and extension view of the spine as evidenced by radiographic evaluation, e.g., one or more of plain X-ray, dynamic X-ray, fine cut bony sequence CT to look for bone crossing the implant (cages) or radiolucency around the screw which is an indirect sign of loosening;
(d) observation after a minimum of 2 months of very limited residual bony activity around the implant and no uptake tracer activity on the bone scan+SPECT; and/or
(e) vertical bone loss around the fixtures should be less than 0.2 mm per year after first year of implant loading.

It will be understood that one or more of the above indicia for osseointegration resulting from use of an implant of the invention compared to an equivalent conventional implant will be obtained in a shorter timeframe than that for the conventional implant, and/or will evidence more clinically relevant osseointegration when the implant of the invention and a conventional implant are subject to the same healing time.

In one aspect, the present disclosure relates to a modular bone anchor. In particular, the modular bone anchor is a pedicle screw for use with bone anchoring to a vertebra. For example, in one embodiment, the modular bone anchor may comprise a pedicle screw system comprising a screw body, a tulip head configured to accommodate a spinal fixation rod, and a set or lock screw suitable for, configured for and/or adapted for use with the pedicle screw of the invention.

The inventors have found that bone stimulation, that is, bone growth and/or strengthening, and/or osseointegration, is facilitated by using an implant having an associated bone stimulation means (ideally, where the bone stimulation means is internally disposed within the implant) that actively but gently loads the bone implant interface thereby providing direct stimulation of bone growth and/or strengthening, and/or osseointegration into the bone/implant interface. Preferred implants have an internal mechanism, preferably a dynamically mobile internal mechanism, that actively but gently loads the bone implant interface thereby providing direct stimulation of bone growth and/or strengthening, and/or osseointegration into the bone/implant interface. In the case of pedicle screws for spinal fixation, this allows application of a much gentler force than occurs when the significant loads are placed on the bone/implant when spinal rod is attached to the pedicle screw which risk overload and poor osseointegration. The implant of the invention is advantageous as is places an effective amount of gentle force required for osseointegration to the bone compared to the zero forces applied when a simple unloaded implant is used or to the significant forces applied when an implant is immediately fully loaded prior to osseointegration taking place. The greater the bone density, the better the chances of clinically relevant osseointegration. It will be appreciated that certain weak bone may require a bone growth and/or strengthening treatment prior to implantation with a load bearing implant for which osseointegration is desirable. The bone growth and/or strengthening treatment can be achieved using the dissolvable implant of the invention as described herein.

Preferably, the bone implant is a polymer implant, a metal implant, a ceramic implant or a combination of polymer, metal and/or ceramic materials. More preferably, a biomedically acceptable material including a metal, such as titanium or stainless steel, a mineral for example apatite, a plastic, a ceramic and combinations thereof.

Desirably, in one aspect, the bone implant may be a pin, a screw used to compress bone fractures, or screws or hooks and the like for supporting a load bearing function, for example, to treat osteoarthritis, scoliosis, spinal stenosis, and chronic pain, etc., a dental implant, a bone-anchored hearing aid, a spinal fusion implant, or an endo-exo prosthesis. A preferred bone implant of the invention is a bone screw, particularly, a pedicle screw.

Suitably, the bone stimulation dynamic interaction, that is, bone growth and/or strengthening, and/or osseointegration promoting dynamic interaction, results from at least one osseointegration-promoting force, provided to at least one area of a bone-implant interface formed when the implant is inserted into bone.

The bone stimulation dynamic interaction, that is, bone growth and/or strengthening, and/or osseointegration promoting dynamic interaction, of the invention can be applied to the bone-implant interface in a continuous, intermittent, periodic or random manner, or in a sequence comprising a combination of in a continuous, intermittent, periodic or randomly applied dynamic interactions.

Desirably, wherein bone stimulation dynamic interaction, that is, bone growth and/or strengthening, and/or osseointegration promoting dynamic interaction is energy in the form of a force, such as a physical force, it may be for example generated mechanically, magnetically or electrically, preferably mechanically. The force is one that results in the described dynamic interaction effects at the bone-implant interface. Preferably, at least one of the forces sufficient to result in local stimulation of cellular responses associated with bone growth and promote bone stimulation dynamic interaction, that is, bone growth and/or strengthening, and/or osseointegration promoting dynamic interaction compared to bone stimulation dynamic interaction, that is, bone growth and/or strengthening, and/or osseointegration promoting dynamic interaction occurring under equivalent conditions with respect to a conventional implant which does not include the at least one means for providing at least one bone stimulation dynamic interaction, that is, bone growth and/or strengthening, and/or osseointegration promoting dynamic interaction as described herein.

Examples of a suitable energy in the form of a force include one or more of pulsing force, a vibrational force, a translational force, a rotational force, a flux force, a frictional force, an expansion force, a contraction force, a pressure force, an repulsive force, an impact force, an electromagnetic force, and an electrical force. It will be understood that more than one of these forces may be present and in such a case, the effect of each force will add cumulatively to provide a net bone stimulation dynamic interaction, that is, bone growth and/or strengthening, and/or osseointegration promoting dynamic interaction, being applied to the at least one area of a bone-implant interface. The action of the net bone stimulation dynamic interaction, that is, bone growth and/or strengthening, and/or osseointegration promoting dynamic interactions or forces provides the described bone stimulation on the bone-implant interface.

In one embodiment, the means for providing the bone stimulation dynamic interaction, that is, bone growth and/or strengthening, and/or osseointegration promoting dynamic interaction, comprises electrodes arranged to provide electrical stimulation to the at least one area of a bone-implant interface.

In another embodiment, the means for providing at least one bone stimulation dynamic interaction, that is, bone growth and/or strengthening, and/or osseointegration promoting dynamic interaction, comprises an acoustic or ultrasound generator/transducer system arranged to provide vibrational energy to the at least one area of a bone-implant interface.

In another embodiment, the means for providing at least one bone stimulation dynamic interaction, that is, bone growth and/or strengthening, and/or osseointegration promoting dynamic interaction, comprises an electromagnetic field generator arranged to provide magnetic field and/or electrical field stimulation/forces to the at least one area of a bone-implant interface. Suitably, the electromagnetic field generator produces electromagnetic radiation in the form of pulsed or low level electromagnetic radiation, for example, at about 5 Hz to about 30 Hz.

In another embodiment, the means for providing at least one bone stimulation dynamic interaction, that is, bone growth and/or strengthening, and/or osseointegration promoting dynamic interaction, comprises a compression/decompression means for providing pressure stimulation/forces to the at least one area of a bone-implant interface. Suitably, the compression/decompression means can generate a variable pressure for providing active pressure stimulation to the bone-implant interface.

In another embodiment, the means for providing at least bone stimulation dynamic interaction, that is, bone growth and/or strengthening, and/or osseointegration promoting dynamic interaction, comprises a mechanical means having at least one moving part arranged to transfer kinetic energy associated with the moving part to the at least one area of a bone-implant interface. It is well understood that kinetic energy is the energy of motion associated with a moving object. The kinetic energy associated with a moving object or part can be transferred and transformed into other kinds of energy. For example, when the implant comprising the mechanical means is moved, potential energy associated with the moving parts of the mechanical means associated with the implant experiences is converted to kinetic energy on experiencing this movement. When the moving part collides with a wall or surface of the implant, the kinetic energy associated with the moving part is transferred to the bone-implant interface as a result of the collision, for example, in the form of heat and vibration.

In another embodiment, the means for providing the at least one bone stimulation dynamic interaction, that is, bone growth and/or strengthening, and/or osseointegration promoting dynamic interaction, comprises at least one mechanism arranged to transfer potential energy associated with the mechanism when loaded to the at least one area of a bone-implant interface. It will be understood that the mechanism is one that can be loaded to form a loaded mechanism, for example, a deformable or resiliently biased mechanism, for example, associated with a coil or a spring. Where the loaded mechanism is provided internally of the implant, it will be understood that the mechanism is an internally loaded mechanism. In another embodiment, the means for providing the at least one bone stimulation dynamic interaction, that is, bone growth and/or strengthening, and/or osseointegration promoting dynamic interaction, comprises an internally loaded mechanism arranged to provide collision stimulation/forces to the at least one area of a bone-implant interface. In one embodiment, the one or more means for providing the bone stimulation dynamic interaction, that is, bone growth and/or strengthening, and/or osseointegration promoting dynamic interaction, is removable from the implant. In some embodiments, a removable means is preferred as it can be added to the implant by the surgeon after initially placement in the bone and does not interfere with the implantation procedure. Likewise, when sufficient bone stimulation has been achieved, a removable means is desirable since it can facilitate further procedures involving loading of the implant.

In one embodiment, the one or more means for providing the bone stimulation dynamic interaction, that is, bone growth and/or strengthening, and/or osseointegration promoting dynamic interaction, is fixed within the implant and cannot be readily removed from the implant.

Suitably, the mechanical means comprises one or more loading mechanisms for providing active load stimulation to the bone-implant interface. By active load stimulation, it is meant stimulation in the form of energy transfer to the bone-implant interface arising from transfer of kinetic energy or energy associated with the momentum of a moving part such as one or more sliding weights or one or more rolling elements associated with the implant. In one embodiment, the mechanical means is one or more actuatable elements associated with the bone implant. Desirably, the one or more actuatable elements are arranged to provide active load stimulation to the bone-implant interface.

Desirably, the mechanical means, for example, the one or more actuatable elements, may be configured to move within or along the implant. Suitably, the one or more actuatable elements are disposed internally within the bone implant.

Preferably, the mechanical means, for example, the one or more actuatable elements, are configured to move with the bone implant in a manner that is dependent on kinetic movement of a patient or subject having the implant. Alternatively, movement of the mechanical means, for example, the one or more actuatable elements, can be independent of kinetic movement, for example, can be controlled by a motor or other controlling device. In one embodiment, movement of the mechanical means, for example, the one or more actuatable elements, with the bone implant can occurs upon direct stimulation, for example, magnetic stimulation, where the mechanical means comprise at least one magnetic material.

Suitably, the one or more actuatable elements are associated with one or more of the implant body, the implant head and/or a removable implant head extension adapted to connect with, or fit over, the implant head.

For example, the implant head may be in the form of a mobile implant head that is adapted to moveably engage with the implant body. The mobile implant head in this case is not an integral part of the implant, but can translatably engage with the implant body such that the implant head is actuatable in and out, or on and off the implant body. For example, in the case of a pedicle screw, the implant head may be a tulip head supported on an elongate body which is dimensioned to fit within the implant body or to fit around the outside diameter of the implant body in a slidable relationship, where the head can slide in and out, or on or off the implant body as the subject moves. In another example, the one or more actuatable elements are associated with the removable implant head extension, for example, the one or more actuatable elements may be provided in the removable implant head extension, whereby the forces generated in the removable implant head extension are transmitted and/or translated through the implant head and implant body to the implant-bone interface. The removable implant head extension can be any shape or dimension that allows association with the implant body and/or implant head. For example, the removable implant head extension can be cylindrically shaped, or can be a conical shape where one side of the head extension has greater diameter than the opposing part of the head extension. The removable implant head extension can be provided on the end of for example a K-wire which can be used with a cannulated implant body of the invention. A preferred removable implant head extension has a distal portion which has a larger diameter than the diameter of the implant head to facilitate location of the implant under the skin for example where the implant is placed as part of a two-step surgical method as described herein. In the case of a conical removable implant head extension, such a larger diameter distal portion is advantageous not only with respect to location of the implant but also acts as a guide for insertion of a K-wire into the implant body, for example, in a procedure where bone cement or graft is required to be provided in the implant body.

In one embodiment, the one or more actuatable elements comprise one or more discrete bodies, for example, solid or hollow particles, grains, rods, and/or rolling or ball bearings, that are freely mobile within the implant body, implant head and/or a removable implant head extension. On movement of the implant, the discrete bodies actively apply a dynamic load and vibration to at least one area of a bone-implant interface formed when the implant is inserted into bone. Preferably, the one or more actuatable elements are provided internally within the implant body. For example, one or more of the osseointegration promotion means can be provided within an internal chamber of the bone implant.

Another actuating element comprises one or more translatable rods, pins, cylinders, or combinations thereof. Suitably, these elements, for example cylinders, are freely mobile within the implant, implant body, implant head and/or a removable implant head extension such that on experiencing movement, the elements translate, for example, longitudinally, transversally, and/or radially within the implant body, implant head and/or a removable implant head extension to provide an active load and mechanical vibration to at least one area of a bone-implant interface formed when the implant is inserted into bone. In one embodiment, where a single cylinder or rod is provided, it is preferred that the cylinder or rod is cannulated, for ready insertion of K-wire and the like.

In one embodiment, at least one of bone stimulation dynamic interaction, that is, bone growth and/or strengthening, and/or osseointegration promoting dynamic interaction means may emit a sonic vibration/frequency that stimulates osseointegration across the bone-implant interface. In another embodiment still, the means, e.g., elements or cylinder, can be translated by the external induction of a current or electrical stimulation device to facilitate active internal load translation.

Suitably, the bone implant further comprises at least one chamber, region, passageway, cannulation or orifice comprising one or more of the means for providing at least one osseointegration-promoting dynamic interaction.

Preferably, the bone implant comprises one or more of internal chambers which accommodate the at least one of the means for providing the bone stimulation dynamic interaction, that is, bone growth and/or strengthening, and/or osseointegration promoting dynamic interaction, to at least one area of bone-implant interface formed when the implant is inserted into bone.

Preferably, the bone implant further comprises one or more apertures or orifices in the implant body. Preferably, the apertures or orifices are adapted to permit bony in-growth into the bone anchor sleeve. Suitably, a plurality of apertures or orifices are provided in the implant. In addition, such apertures or orifices can adapted or configured to retain and pass bone cement, osteo-inductive and/or genetic materials (e.g., BMP) to facilitate the augmentation of bone-implant fixation and osseointegration.

A preferred bone implant of the invention comprises an implant body for insertion and retention of the implant into bone, an implant head engagable with a tool for insertion of the implant body into the bone. In some embodiments, the bone implant can be provided with a neck portion for connecting the implant body with the implant head. In a preferred embodiment, the implant head is a tulip head or the like for holding or retaining a spinal rod for example. Suitably, the implant may further comprise an implant set or locking screw or cap adapted to connect to the implant head, for example, a tulip head, so as to secure a spinal rod in place within the tulip head.

Desirably, the bone implant comprises one or more threads for insertion, engaging and/or retention into bone. Suitably, the screw can be a self-tapping or a non-tapping screw. An exemplary bone implant is a bone anchor being a dental implant, or a bone screw. An implant with a thread allows for the conversion of rotational forces into linear motion to drive or advance the implant into bone. Depending on the bone desired for insertion the dimensions, particularly the length and implant body diameter and thread shape, pitch and depth of the screw can be adapted as required. Suitably, the surgical screw is a cortex screw, a cancellous screw, or a pedicle screw. Most preferably, the bone implant is a pedicle screw which is one example of a spinal fusion implant. It will be understood that on experiencing torque, the head contacts bone generating compression under the head and tension in the implant shaft whereby friction between the bone and the thread of the inserted implant caused produces primary stability.

A preferred bone implant comprises an implant body cover for closing off internal parts of the implant body for example to prevent tissue or bony ingrowth into the implant body. Preferably, the implant body cover is a temporary cover, such as, a perceivable cover, for example, a frangible or otherwise deformable cover, such as a pierceable plastic or other deformable material which is suitable for piercing, for example, for recannulation of the implant at a later time. A particularly preferred implant body cover is adapted for easy of location after the implantation surgery has occurred. For example, the implant body cover may have a shape or configuration that enables the cover to be ready located under the subject's skin, for example, through tactile examination. For example, the implant body cover may comprise a pointed or textured surface for tactile location under the skin. For example, the implant body cover may be conical in shape with the pointed end towards the skin, or may be provided with indentations, ridges, nubs or other textural features which facilitate tactile location under the skin.

Suitably, the bone implant can be adapted for open surgical, a minimally invasive surgical technique (MIS) or other percutaneous application.

Desirably, the bone implant further comprises surface functionalisation, for example, at least one bioactive coating such as a bone mimetic coating, a drug eluting coating, an anti-inflammatory coating, and an anti-microbial coating.

Desirably, the surface of the implant can be provided with a coating for promoting a structurally stable interface between the implant and the surrounding bone. Suitable coatings include hydroxyapatite or hydroxyapatite derivatives. Desirably, the surface of the implant can be treated to promote osseointegration. In one embodiment, the bone implant may further comprise additional chemical or biomedical means for promotion of osseointegration and/or faster healing time, and/or reduced risk of infection.

Preferably, the bone implant is provided with, or is adapted to function with, vital biological cues, growth factors and cells in order to improve osseointegration and repair of bone defects. For example, the implant may be used in conjunction with one or more of a bone graft and/or an osteoinductive scaffold, steroids, simvastatin, insulin-like growth factor (IGF), bone morphogenetic protein (BMP), vascular endothelial growth factor (VEGF), platelet-rich plasma (PRP), TGF-β, platelet-derived growth factor (PDGF), antibiotics, etc.

Desirably, the implant is provided with at least one porous region at which angiogenesis and/or bony ingrowth can be facilitated.

Suitably, the surface of the implant can be roughened. Roughened or etched surfaces have been found to facilitate osseointegration.

Suitably, the bone implant comprises one or more biomedically acceptable materials. Preferred biomedically acceptable materials are one or more of biocompatible, osteoconductive, osteoinductive, enable angiogenesis, bioresorbable/biodegradable, and nontoxic. Particularly, preferred biomaterials are those considered as an alternative to natural bone with similar osteoinduction, osteoconduction, inflammation and mechanical integrity as native bone. Suitably, the implant or one or more parts of the implant is formed by 3D printing.

Depending on a desired function the biomedically acceptable material may be biologically active (bioactive) or biologically inert (bioinert). Bioinert means the material does not initiate a response or interact when introduced to biological tissues. If bioactivity is required, the implant can be provided with an appropriate coating, for example a bioactive coat of hydroxyapatite.

Preferred biomedically acceptable materials include metallic biomaterials, bioceramics, biocomposites, polymers and combinations thereof. Implants for bone repair and other load bearing implants require high mechanical strength, and for these applications, bioceramics and metallic biomaterials are preferred.

Examples of metallic biomaterials include biomedically acceptable metals and metal alloys. For example, cobalt chromium, tantalum, stainless steel, zirconium, magnesium and magnesium alloys such as Mg—Zn-based, Mg—Ca-based, Mg—Si-based, Mg—Sr-based and Mg-rare earth alloys, iron alloys, titanium or titanium alloys, and in the case of orthopaedic implants, chromium, cobalt, molybdenum, nickel, titanium and zirconium alloys. Titanium alloys are particularly preferred as they exhibit considerably superior biocompatibility compared to their other counterparts due to their excellent corrosion resistance and form easy bonding with bone, demonstrating good integration with bone tissue.

Examples of bioceramics include zirconia and alumina based ceramics, calcium phosphate ceramics, hydroxyapatite (HAp), hydroxyapatite (HAp) derivatives including strontium, zinc, magnesium and silicon doped hydroxyapatites, dicalcium phosphate dihydrate (DCPD), brushite, monetite, and tricalcium phosphate (TCP)

Examples of polymers include polycyanoacrylates, polyanhydrides, poly(amino acids), poly(ortho ester), polyphosphazenes , poly(propylene fumarate), polylactic acid (PLA), poly glycolic (PGA) and copolymers, polyhydroxybutyrate (PHB), polyhydroxyvalerate (PHV), and copolymers, polycaprolactone, and polydioxanone. Preferred polymer include starch, chitosan, hyaluronic acid derivatives, collagen, fibrin gels, silk, with and without induced crosslinking, polyglycolic acid (PGA), polylactic acid (PLA), poly-β-hydroxybutyrate (PHB), poly (lactic acid-co-glycolic acid) (PLGA) and poly-ε-caprolactone (PCL).

A particularly preferred biomedically acceptable material is a bioresorbable/biodegradable biomedically acceptable material, which is one that dissolves itself to replace the surrounding tissues. Bioresorbable bioceramics are designed to gradually degrade in a time frame followed by replacement of natural host tissue. Bioresorbable bioceramics are well known in the art.

In one particularly preferred embodiment, the bone implant is dissolvable in situ. A transient bone implant is particularly desirable as it does not need to be removed by a surgical technique. This is advantageous because of the risks associated with medical implants which include surgical risks during removal, infection and implant failure. Further, certain patients have reactions to the materials used in implants e.g. an allergic foreign body response. Depending on the materials used in the construction of the device, the bone implant can stably sit in position for a predetermined timeframe, for example, weeks or months. Dissolvable in situ implant are particularly relevant in the context of embodiments described herein relating to bone growth, strengthening, densification of weak bone, for example, osteoporotic or osteopenic bone.

In addition to selection of a suitable bioresorbable material, the implant properties with regards to porosity, pore size, and pore interconnectivity can be tailored towards preferred rates of degradation, osteoconductive, osteoinductive and/or angiogenesis capability, For example, porous magnesium alloys can be used where rapid in vivo degradation is require, while higher porosity aids in angiogenesis.

A particularly preferred implant comprises a longitudinally extending bone anchor sleeve having a proximal end and a distal end.

Suitably, the implant may comprise one or more passageways extending between the proximal and distal ends. In one embodiment, the passageway may be in the form of a cannulation.

Preferably, the implant comprises a first engagement formation located on a radially outer surface of the bone anchor sleeve.

Desirably, the implant comprises an internal member having a head and a shaft, wherein the shaft is configured to be seated within and secured relative to the passageway such that the head is positioned adjacent to the proximal end of the bone anchor sleeve.

In a related embodiment, the invention provides a bone anchor comprising:

a longitudinally extending bone anchor sleeve having a proximal end and a distal end, a passageway extending between the proximal and distal ends,

a first engagement formation located on a radially outer surface of the bone anchor sleeve; and

an internal member having a head and a shaft, wherein the shaft is configured to be seated within and secured relative to the passageway such that the head is positioned adjacent to the proximal end of the bone anchor sleeve.

Suitably, the bone anchor further comprises at least one means for providing at least one osseointegration-promoting dynamic interaction as described herein to at least one area of a bone-anchor interface formed when the implant is inserted into bone.

In one example, the anchor as described herein further comprises one or more apertures or orifices extending distally between an outer surface and the internal passageway.

Preferably, the anchor further comprises a plurality of apertures, for example, extending distally between an outer surface and the internal passageway, the apertures adapted to permit bony in-growth into the bone anchor sleeve. In addition, such apertures can be utilised in such a manner as to retain and pass bone cement, osteo-inductive and genetic materials (e.g., BMP) and therefore facilitate the augmentation of bone-implant fixation and osseointegration.

In another aspect, the invention provides for use of the bone implant of the invention as a device for treating and/or preventing a bone wasting/weakening disease or disorder such as osteopenia and/or osteoporosis.

In another aspect, the invention provides a use of a bone implant of the invention in combination with one or more bone grafting techniques including use of autograft, allograft, bone graft substitutes such as hydroxyapatite, tricalcium phosphates, biphasic calcium phosphates, etc.

In another aspect of the invention, there is provided a use of the bone implant described is used in a surgical technique. For example, the bone implant of the invention may be used in the treatment, prevention and/or correction of one or more of spinal stenosis, spondylolisthesis, spinal deformities, fracture, pseudoarthosis, tumour resection, failed previous fusion., degenerative disc disease, dislocation, scoliosis, and kyphosis.

Suitably, the bone implant may be adapted use in for open surgical and/or minimally invasive surgical (MIS) insertion into bone. For example, the implant may comprise one or more cannulations from accommodating guide wires and the like used in a MIS technique.

In a preferred embodiment, the implant is adapted to facilitate location and assessment via an analytic or diagnostic technical, for example, an imaging technique, which can be a radiological technique, for example CT, or fluoroscopy, or an MRI investigative means. Intra-operative techniques are preferred.

While certain implants, for example, hip implants, are intended to be permanent, bone implants such as screws used to repair broken bones can be removed when they no longer needed or there may be been misplacement of the implant during surgery or it may be required to repair or replace an implant which has moved, broken, or stop working correctly, infection, inflammation, pain. Desirably, the implant can be adapted to be readily removed through non-invasive or minimally-invasive techniques which preferably can be carried out without anaesthetic or under local anaesthetic.

In another aspect, the invention provides a method of promoting stimulation including bone growth, strengthening and/or densification comprising implanting into a subject in need thereof, an implant according to the first to third aspects of the invention.

In another aspect, the invention provides a method of promoting osseointegration comprising implanting into a subject in need thereof, an implant according to the first to third aspects of the invention.

In another aspect, the invention provides a surgical method comprising the step of inserting the bone implant of the invention into a bone. Suitably, the surgical method comprises the step of making an incision in the skin and supporting tissues at a site where the implant is to be inserted. It will be understood that the surgical method further comprises the step of closing the incision in the skin and supporting tissues. Suitably, the incision in the skin and supporting tissues is sufficient to enable visualisation of the bone. Visualisation of the bone may be direct visualisation by eye or may be a radiographic visualisation or a CT visualisation or a combination thereof.

In embodiments where the implant is used for bone stimulation including enhancing bone growth, strength and/or osseodensification, a single step surgical procedure is sufficient, particular where the implant used is a dissolvable implant as described herein.

Desirably, the surgical method is a two stage surgical method involving a first stage surgical intervention whereby the bone implant of the first, second or third aspects is inserted into a bone at a first time point, and a second stage surgical intervention whereby a load is applied to the bone implant inserted in the first stage surgical intervention at a second time point. The two stage surgical method is particularly desirable where the indication requires a load to be placed on the implant, for example, spinal fixation with instrumentation where the load applied is in the form of a spinal rod, for example. Equally, the method could apply to a prosthesis implant whereby an artificial arm or leg for example is added to the implant.

Suitably, the intervention associated with the first time point and the intervention associated with the second time point occurs at an interval sufficient to allow an acceptable level of osseointegration to occur between the implant and bone. Suitably, the intervention occurring at the first timepoint and the intervention occurring at the second time point are spaced apart in time, and for example, occur at an interval of at least 2 weeks, more preferably 4 weeks, 12 weeks, 24 weeks or longer. The interval length can be assessed on the basis of one of the osseointegration as described herein.

In another aspect, the invention provides a kit of parts comprising a housing for accommodating a plurality of the implants of the invention aspects. For example, a preferred housing is a caddy, for example, a metal or a plastic caddy where by the metal or plastic material from which the caddy is formed is sterilisable. The kit may further comprise instrumentation for carrying out a particular surgical procedure, for example, a spinal fixation system. In a particularly preferred embodiment, the kit comprises the implant of the invention, adapted to include a tulip head for accommodating a spinal rod, and a set or lock screw head for maintaining the rod in position in the tulip head.

In another aspect, the invention extends to the use of the implant of the invention in conjunction with an osteogenesis strategy and/or a tissue engineering strategy, for example, a bone graft and/or an osteoinductive scaffold. The tissue engineering strategy may further include one or more of the following components: steroids such as dexamethasone; simvastatin which has been shown to enhance bone formation in particular by promoting the differentiation of cells into the osteogenic lineage; insulin-like growth factor (IGF) which is well known for promoting osteogenic cell differentiation and being involved in the regulation of several key cellular processes such as proliferation, movement, and inhibition of apoptosis; bone morphogenetic protein (BMP), in particular, BMP-2, which is known to play an essential role in bone healing by influencing osteogenesis as well as vascularization. Bone morphogenetic protein (BMP) is especially effective when combined with stimulation with pulsed electromagnetic fields; vascular endothelial growth factor (VEGF) is by excellence the family of molecules responsible for vascularization; platelet-rich plasma (PRP) which provides a gel-like physical support and drug delivery vehicle and which is composed of growth factors such as TGF-β as well as platelet-derived growth factor (PDGF) which is a crucial promoter of bone healing involved in the initiation of callus formation and in angiogenesis; antibiotics, for example, vancomycin, an anti-gram positive bacteria drug which can be integrated into implantable materials in various forms.

The osteogenesis strategy may involve osteogenesis stimulation for example, chemical or biophysical osteogenesis stimulation, e.g., biophysical osteogenesis stimulation can include one or more of electrical stimulation though application of electrodes to the bone to be treated; capacitive systems involving the application of electric fields on the bone; application of ultrasound to produce mechanical vibrations on the bone; inductive systems, pulsed electromagnetic fields which product a magnetic field that induced an electric field on the bone.

In another aspect, the invention provides a bone implant comprising at least one means for providing at least one bone growth, bone strengthening, and/or bone densification dynamic interaction to the bone surrounding the bone-implant interface.

In another aspect, there is provided a bone implant comprising at least one means for providing at least one osseointegration-promoting dynamic interaction to at least one area of a bone-implant interface formed when the implant is inserted into bone.

In another aspect, there is provided an implant body cover for a bone implant for closing off internal parts of the implant body, preferably a temporary cover, such as, a perceivable cover, for example, of a frangible material suitable for recannulation of the implant. For example, the implant body cover may have a shape and/or configuration that enables the cover to be ready located under the subject's skin, for example, through tactile examination. A preferred implant body cover has a conical shape and/or configuration shape adapted such that, in situ, the conical sharp end is located towards the skin.

In another aspect, there is provided a kit of parts comprising a plurality of the implants of the invention. Preferably, the kit further comprises a plurality of the implant body covers of the invention. Preferably, the kit further comprises one or more housings for accommodating the plurality of the implants and/or the plurality of the implant body covers. Desirably, in one embodiment, the kit is in the form of a surgical instrumentation kit for a spinal fixation surgical procedure.

### BRIEF DESCRIPTION OF THE DRAWINGS

A preferred embodiment of the invention will now be described by way of specific example with reference to the accompanying drawings, in which:

FIG. 1 is a schematic view of a partially sectioned vertebra including two prior art pedicle screws;

FIG. 2 illustrates a cannulated pedicle screw comprising multiple free moving metallic balls;

FIG. 3 illustrates a similar embodiment to that of FIG. 2 although the multiple free moving metallic balls have been replaced with a single load in the form of a weighted cylinder; and

FIG. 4 illustrates the use of an external magnetic field to increase the displacement of the load/loads located within the inside chamber of the screws detailed in FIGS. 2 and 3.

FIG. 5(a) to (c) illustrates further preferred embodiments of the invention; (a) is a pedicle screw and tulip head arrangement with one example of a removable implant head extension with freely mobile particles within the implant head extension of the invention; (b) is a pedicle screw shank and a mobile, slidable implant head of the invention; (c) is a cannulated pedicle screw with conical shaped removable implant head extension, the extension having a temporary anti-tissue overgrowth cap.

### DESCRIPTION OF THE INVENTION

### Healing Process and Bone-Implant Integration

Healing and bone integration is a complex process. Insertion of the screw causes disruption of the normal bone structure and results in the formation of a blood clot around the insertion site. The blood clot attracts blood cells to clean up the wound, initially monocytes then macrophages, as it the case for any wound. These cell, especially the macrophages, produce growth factors which promote angiogenesis and activate fibroblasts to produce an extracellular matrix. The “tissue” produced at this stage is called granulation tissue. Weak bone called woven bone or fibrous bone then results. The bone will be produced first around the new blood vessels, explaining why the local environment is so important (for example smokers have been shown to have poor bony healing because of the toxins in the cigarette affecting newly formed blood vessels). Mesenchymal progenitor cells then differentiate into osteoblasts cells producing new bone and the woven bone soon bridges between the implant or screw, for example, and the host bone. If the distance is too large this process cannot happen and this explains why it is important to have good initial contact between the implant and the host bone. The next step involves formation of stronger bone called lamellar bone and only after, bone remodelling (which is influenced by loading and stress) occurs. Wolff's law holds that bone adapts to functional loading. For example, if loading on a particular bone increases, the bone will remodel itself over time to become stronger to resist that sort of loading. Likewise, if the loading on a bone decreases, the bone will become less dense and weaker due to the lack of the stimulus required for continued remodelling. However, the inventors believe that too much loading prevents optimal osseointegration and not enough (e.g., a screw left alone in the bone completely unloaded) loading negatively affects osseointegration. The devices described herein, particularly those with internal loads, produce an adequate level of stimulation of the surrounding cells to achieve optimal osseointegration within the best timeframe. After sufficient time, when optimal osseointegration is achieved as a result of utilisation of the device of the invention, the screws and osseointegrated bone can readily and better sustain the required loading produced by, for example, rod insertion, compared to the performance where conventional screws are used and are loaded immediately after surgical implantation.

### Evaluation of Osseointegration

There are several ways of assess bony healing of an implant. One method involves a clinical assessment. A solid and/or secure implant should not cause any pain or instability (pain or neurological deficit during movement). Another method of confirming bony healing is radiologically, using for example, plain X-rays, dynamic X-rays (there should not be any movement at the level fused during the flexion and extension view of the spine) and/or fine cut bony sequence CT to look for bone crossing the implant (cages) or lucency around the screw which is an indirect sign of loosening. Alternatively after a few months there should be very limited residual bony activity around the implant and therefore there should not be any uptake tracer activity on the bone scan+SPECT. In some cases, the assessment must be made by exploratory surgery.

There are several other methods for the evaluation of the degree of osseointegration, broken into invasive and non-invasive categories. Invasive methods include histological section, histomorphometric, transmission electron microscopy, pull out tests, torque gauge tests. Microscopic or histologic analysis has been considered as the gold standard method to evaluate the degree of osseointegration. However, due to the invasiveness of this method and related ethical issues, various other methods of analysis have been proposed. Suitable non-invasive methods include light microscopy, microcomputed tomography, backscatter electron imaging, percussion test, radiographs, reverse torque test, periotest, and resonance frequency analysis.

In the percussion test, an osseointegrated implant will be found to make a ringing sound on percussion whereas an implant that has undergone fibrous integration produces a dull sound.

In the torque gauge test, a reverse or unscrewing torque is applied to assess implant stability at the time of abutment connection. Implants that rotate under the applied torque are considered failures and are then removed.

The periotest relates to a device which is an electrically driven and electronically monitored tapping head that percusses the implant a total of 16 times in about 4 s.

Scanning electron microscopic study of the interface typically shows the characteristic absence of connective tissue between the bone and implant surface.

Resonance frequency analysis measures implant stability and bone density at various time points using vibration and structural principle analysis. Classically, the implant stability quotient (ISQ) has been found to vary between 40 and 80, the higher the ISQ, the higher the implant stability. It is inversely proportional to the resonance frequency. Implant stability can be determined for implants with an ISQ of 47. All implants with an ISQ more than 49 osseointegrated when left to heal for 3 months. All implants with an ISQ more than 54 osseointegrated when immediately loaded.

Osseointegration may also be assessed by applying the Alberktsson Success Criteria: 1. The individual unattached implant should be immobile when tested clinically. 2. The radiographic evaluation should not show any evidence of radiolucency. 3. The vertical bone loss around the fixtures should be less than 0.2 mm per year after first year of implant loading. 4. The implant should not show any signs of pain, infection, neuropathies, parasthesia, violation of mandible canals and sinus drainage. 5. The success rate of 85% at the end of 5 year and 80% at the end of 10 years.

### Staged Implantation Technique

The implant of the invention can be used in open surgery, minimally invasive surgery. Desirably, implant can be used in a staged implantation technique whereby in a first stage, the implant is positioned and given time for a desirable amount of osseointegration to occur.

After the surgical placement of implants into a desired location, the traumatized bone around the implant begins the process of wound healing which can be separated into an inflammatory phase (involving vascular and cellular events), proliferative phase (involving vascularization, cell differentiation into fibroblasts, osteoblasts and chondroblasts and eventual bone callus formation) and a maturation phase (involving ossification of the bone callus, and bone remodelling).

The staged implantation technique requires a bone implant that comprises a modular distal threaded bone anchor and a proximal screw head, wherein the bone implant comprises one or more of described loading mechanisms, for example, an internal rolling element or cylindrical member. A preferred implantation technique involves the following:
i) Surgical Stage 1—a first bone implant, for example, a bone anchoring component is inserted into a bone, for example, a bone pedicle. This bone anchoring component used is suitably adapted to function in use with one or more of the loading mechanisms required to stimulate osteogenesis. The method preferably further comprises a pre-preparation step involving inserting bone substitute or osteoinductive bone morphogenic proteins (BMPs) into a passageway provided in the implant to facilitate osteoconduction. It will be understood that osteoinduction is the process by which osteogenesis is induced. It is a phenomenon regularly seen in any type of bone healing process. Osteoinduction implies the recruitment of immature cells and the stimulation of these cells to develop into preosteoblasts.
ii) Surgical Stage 2—is preferably separated from Surgical Stage 1 by a period of at least 6 weeks or other period required to ensure a sufficient amount of osseointegration has taken place. Surgical Stage 2 involves removal of the aforementioned osseointegration promotion means for applying a dynamic physical force to bone at one or more bone-implant interfaces, if necessary, followed by insertion of an internal member that completes the bone anchoring component, for example, a poly-axial screw head in the case where the bone anchoring component is a threaded pedicle screw shank, allowing final construct fixation and fusion with a rod.

When used in the staged implantation method the clinician will be able to identify sufficient bony on growth or in growth and subsequently decide to insert polymethylmethacrylate or another suitable biocompatible bonding agent into the passageway.

The benefit of staged implantation allows the clinician to assess after at least a 6 week period, the quality of osseointegration with the bone anchor device. This can be assessed radiologically or by other investigative means. This is of particular utility in patients who are osteopenic, in that the level of adequate integration of the device can be measured before attempting to finalise a construct for fusion. Early fusion of construct without adequate bone anchor integration can result in poor fixation, fusion failures and other complications. The clinician can track and assess the required level of fusion and then at the appropriate time either fuse or revise surgical strategies.

### System—Novel Instrumentation

In a preferred embodiment, the implant includes but not limited to: an in vivo bone density measurement tool (qualitative).

Further instrumentation can be devised, including but not limited to an electronic device that is placed into the stage 1 bone anchoring component that runs a current through the implant and bone and provide feedback and qualitative measurement of the degree of osseointegration. This adaptation provides easy feedback by way of instrumentation for the surgeon.

### Novel Internally Loaded Design for Dynamic Osseointegration

The internal chamber of the threaded shank is unique in that its bone promoting mechanism can be described as follows:
i) One or multiple ball bearings that are freely mobile within the screw shaft that actively load and vibrate the screw/bone interface in order to stimulate osteogenesis (for use in open screw techniques)
ii) A cannulated cylinder that longitudinally translates within the cannulated screw shank that provides an active load and mechanical vibration to stimulate osteogenesis in the screw/bone interface (for use in percutaneous application)
iii) The internal mechanical devices described in i) and ii) could be comprised of magnets that are translated by polarised magnets placed at both proximal and distal ends of the screw shaft.
iv) An alternative to translating the mechanism in iii) is by the external induction of a current or electrical stimulation device to facilitate active internal load translation.
v) An internal cylinder as described in ii) that emits a sonic vibration/frequency that stimulates osseointegration across the bone/screw interface.

### Screw Head

While the implant described herein can be used as a classic pedicle screw, it is believed that using the implant in conjunction with a two-stage implantation and loading procedure would increase significantly the quality of the osseointegration. This means leaving the implants, e.g., screws, inside the host for a sufficient length of time to allow optimal osseointegration to occur as a result of the dynamic loading provided by the implant of the invention (e.g., 2-6 months), prior to applying the functional load, in a second procedure, for example, connect them to a rod in the case of spinal fixation.

In the context of a two-stage implantation, a preferred head for the implants of the invention ideally have specific characteristics to facilitate this use. For example, preferred implant heads are designed to avoid any bony or tissue ingrowth inside them which is undesirable, e.g, resulting in rod interference or prevention of installation of a set screw. A preferred implant head is easy to find. For the most preferred implant heads, re-cannulation without direct visual sight is possible. A preferred implant head has a conical shape that can be re-cannulated regardless of the angle. A preferred implant head has pierceable cap, which can be pierced by a specific instrument, and which can be located via tactile feedback or other kind of feedback to confirm the surgeon that the head was adequately cannulated. The pierceable cap, for example, a plastic cap can desirably also prevent tissue or bone ingrowth inside the head.

Preferred implant insertion involves a minimally invasive approach (e.g., stab incision) under X-ray, or more preferably, CT guidance using a technology such as the intraoperative O'Arm. A similar technique may be used to connect the implant head to the rod, however, the tulip head typically used can be quite difficult to find as the implants are usually quite deep under a thick layer of muscle and/or fat and reattaching towers or inserting a screw driver inside them can be challenging.

### Dissolvable Implant

A dissolvable screw can be inserted using a minimally invasive approach. Once the implant is fully dissolved then the final procedure (e.g., a standard lumbar fusion surgery) would be performed.

A preferred implant has the internal structure described herein, but the shaft and/or internal load could have small balls that are moving freely and will also gradually dissolve. The balls or the shaft is at least partially coated in recombinant bone morphogenic protein which will enable more rapid and more efficiently differentiation of mesenchymal cells into bone forming cells. Furthermore, the implant thread is preferably designed to create enough granulation tissue to enhance bone formation rather than optimal primary fixation, as the implant of this aspect of the invention would not be loaded. The purpose of the dissolvable implant is localized bone structure enhancement. Use of the dissolvable implant in weak bone will, through application of the dynamic forces described herein, change the internal bony structures of a vertebra from normal or weak or cancellous or osteoporotic bone into a stronger bone which would offer a better structure for the subsequent insertion of a load bearing implant such as a pedicle screw. In other words, the dissolvable implant of the invention functions as a localized bone structure enhancing device.

### Description of Preferred Embodiments of the Invention

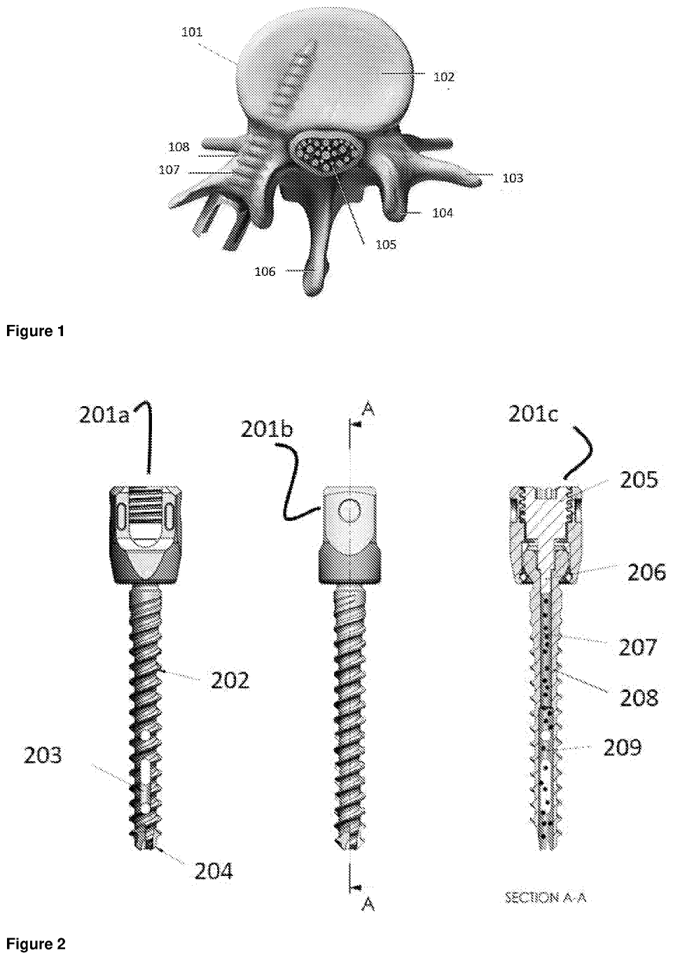

Turning now to FIG. 1, which shows a top down view of a vertebra 101 into which vertebral body 102 is inserted a pedicle screw 107 through the pedicle bone 108 of the vertebra 101.

FIG. 2(a) is a cannulated pedicle screw 201a having a shank 202 and a tulip head and a dummy cap weighted for bone loading provided at one end of the shank. The shank 202 is provided with fenestrations 203 to allow egress of bone cement and the like and/or ingress of bony tissue. The cutting tip geometry 204 is also shown. FIG. 2(b) shows the side profile of the screw of FIG. 2(a), which FIG. 2(c) shows a sectional view of the screw in which the following are illustrated: the dummy cap (removable implant head extension) 205 which comprises a rod portion 206 to lock polyaxial motion during osseointegration; a section view of the cannulation 207; freely moveable elements 208, particles or spheres, inside the cannulation 207 of the screw 201. The wide diameter cannulation 209 is also shown which accommodates the moveable elements 208.

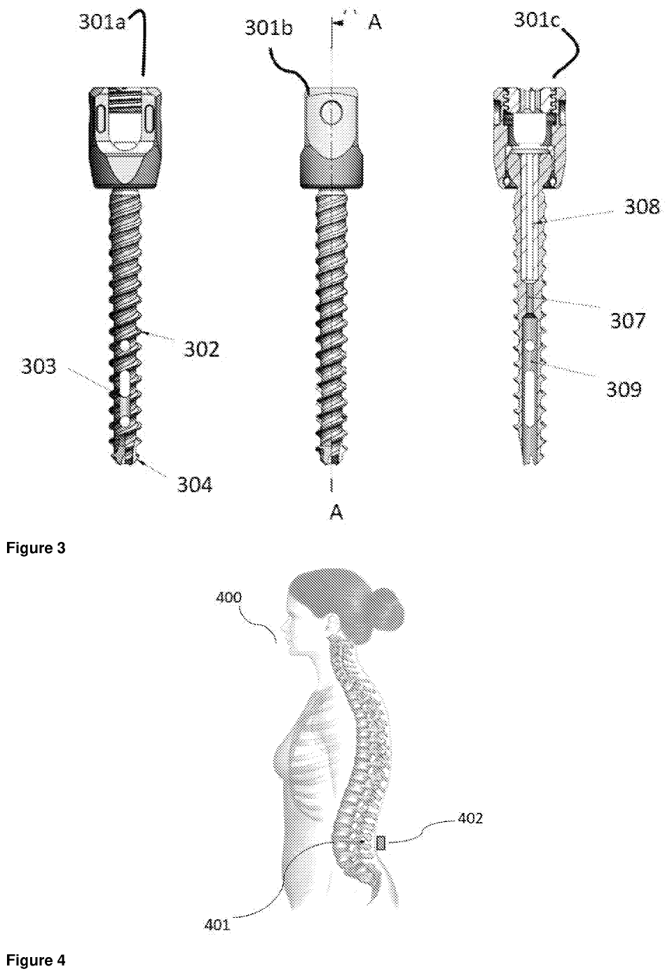

FIG. 3(a) is a cannulated pedicle screw 301a having a shank 302 and a tulip head and a standard locking cap or set screw for locking a rod in place provided at one end of the shank. The shank 302 is provided with fenestrations 303 to allow egress of bone cement and the like and/or ingress of bony tissue. The cutting tip geometry 304 is also shown. FIG. 3(b) shows the side profile of the screw of FIG. 3(a), which FIG. 3(c) shows a sectional view through line A-A of 301b of the screw in which the following are illustrated: the locking cap; a section view of a portion of narrow cannulation 307, and larger diameter cannulation 309; translatable element 308, a single cylinder, inside the implant body 302.

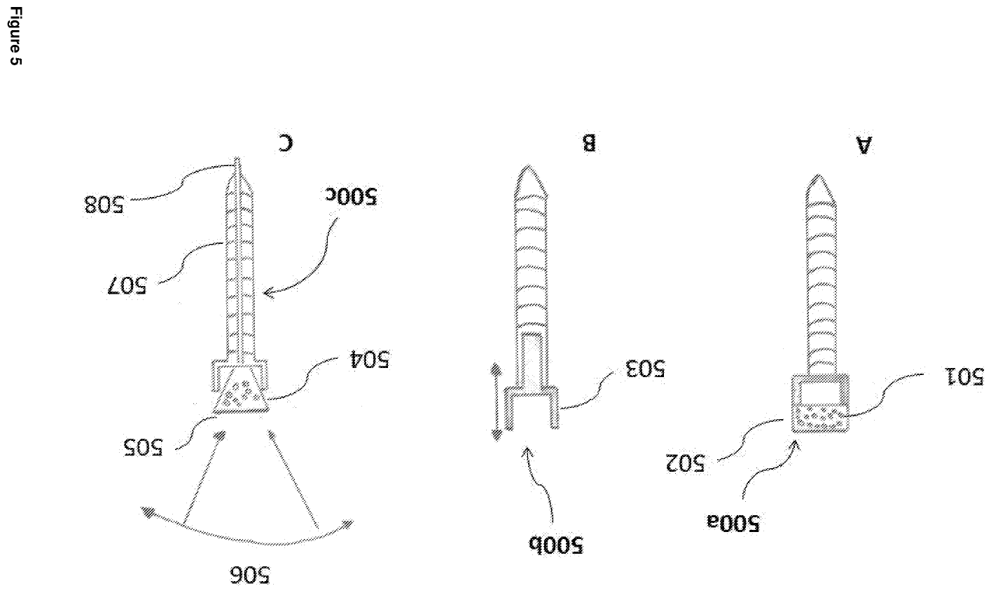

FIG. 4 illustrates a patient 400 having implanted pedicle screw in L4 vertebra 401 and a magnetic field generator 402 for enabling trans cutaneous magnetic stimulation of magnetised actuatable elements within the implant.

FIG. 5(a) illustrates a pedicle screw 500a having a removable implant head extension 501 which comprises internally the actuatable elements, in this case particles 501 which are freely moveable and which resultant kinematic energy or forces is translated through the implant to the bone-implant interface. FIG. 5(b) illustrates a pedicle screw 500b having a mobile implant head 530 that is adapted to moveably engage with the implant body. The mobile implant head can translate back and forth along a portion of the longitudinal axis of the shaft in the directed indicated by the arrows to thereby generate the required mechanical motion to generate the required forces. FIG. 5(c) illustrates a cannulated pedicle screw 500c having a shank 507 and a tulip head and a conically shaped removable implant head extension 504, which in this example contains freely moveable particles. The upper surface of the removable implant head extension is covered with a frangible cover material to prevent tissue ingress or ingrowth within the implant and/or around the upper portion of the implant body where the rod and/or set/lock screws are to be placed, for example, a biocompatible polymer which is pierceable by incision or puncture, with for example, a K-wire for bone cement introduction or other such operation. The conical shape of the head or the extension advantageously allows for a much wider degree of cannulation.

### EXAMPLE 1

One or more rolling elements or bearings, for example, ball bearings, that are freely mobile within the implant body such that on movement of the implant, the rolling elements actively apply a dynamic load and vibration to the bond-implant interface in order to stimulate osteogenesis.

This embodiment is illustrated in FIG. 1 which shows an example of a preferred implant of the invention, that is, a cannulated pedicle screw. The tip of the screw is designed to allow bone ingrowth within the shaft. The internal chamber contains multiple free moving metallic balls. The outside shaft of the bone anchor depicted has a specific thread to ensure maximal pull-out strength or primary fixation. In this example, the pedicle screw has a poly-axial head adapted to engage rods. The poly-axial head is closed with a specific cap preventing tissue ingrowth when the pedicle screw is used as an anchor for example in Stage 1 surgery whilst waiting for osseointegration to occur, and is designed to receive a drive tool such as a hexagonal tool or another such torque transmission tool.

### EXAMPLE 2

A cannulated cylinder that longitudinally translates within the implant body that provides an active load and mechanical vibration to stimulate osteogenesis in the bone-implant interface and is particularly suited for use in a percutaneous application. The internal cylinder can be adapted to emit a sonic vibration/frequency that further stimulates osseointegration across the bone/screw interface.

This embodiment is illustrated in FIG. 2 which shows a similar embodiment to FIG. 1 although the multiple free moving metallic balls have been replaced with a single load. The load has a cylinder configuration and traverses up and down within the inside chamber generating the required forces and/or vibration at the bone-implant interface.

### EXAMPLE 3

The loading mechanisms described in Examples 1 and 2 comprising a magnetic material such that the elements or cylinder can be translated by polarised magnets placed at both proximal and distal ends of the implant. Alternatively, the elements or cylinder can be translated by the external induction of a current or electrical stimulation device to facilitate active internal load translation. This embodiment is illustrated in FIG. 3 which shows the use of an external magnetic field to increase the displacement of the load/loads located within the inside chamber of the screws detailed in FIGS. 1 and 2. The patient applies on his skin at the level of the implant, a magnetic field generator contained in a small ‘portable’ device. The generator delivers an electrical current across the tissue without physical contact increasing the displacement of the loads within the inside chamber of the screw.

### EXAMPLE 4

The example in FIG. 5(a) is a pedicle screw and tulip head arrangement with one example of a removable implant head extension with freely mobile particles within the implant head extension of the invention which overlays a typical tulip head of a pedicle screw. The removeable head extension is placed during an initial surgical step to prevent tissue ingress into the tulip head during healing. The gentle dynamic forces resulting from movement of the actuatable elements in the removeable head extension are translated through the screw head to the implant body and to the implant/bone interface.

### EXAMPLE 5

The example in FIG. 5(b) is a pedicle screw and tulip head arrangement where the actuatable element of the mechanical means is the tulip head itself. The tulip head is mounted on a leg portion which is adapted to fit within the internal circumference of the implant body but it is not locked into this position. Instead the implant head is free to move up and down the longitudinal axis of the implant body in the direction of the arrows shown in the Figure. When sufficient healing has occurred, locking of a spinal rod in the tulip head can cease the head movement.

### EXAMPLE 6

The example in FIG. 5(c) is a pedicle screw and tulip head arrangement where the actuatable element of the mechanical means is provided in a conically shaped removable implant head extension with freely mobile particles provided within the implant head extension. In this example, the conically shaped removable implant head is provided on K-wire to facilitate insertion into the cannulated implant body as shown. While prevent tissue ingress, the wider diameter of the conically shaped removable implant head in the distal portion of the conically shaped removable implant head advantageous allows easier location of the implant under the skin and tissue after healing has occurred, and provides a wider guide for inserting a K-wire or syringe or the like to for example facilitate insertion of bone cement or other materials. In this example, the conical tip can comprise or more apertures to facilitate this insertion of other devices. The conically shaped removable implant head has a temporary and/or frangible membrane cover enabling later removal of the actuatable elements and/or facilitation of insertion of other tools/objects.

1. A bone implant comprising:
an implant body, an implant head, and optionally, an implant head extension for removeable attachment to the implant head, and
at least one mechanical means associated with the bone implant for providing at least one bone stimulation interaction to at least one area of a bone-implant interface formed when the implant is inserted into bone, wherein bone stimulation is one or more of bone growth, bone strengthening, bone densification and osseointegration between and/or around the bone and the bone-implant interface,
wherein the mechanical means comprises one or more actuatable elements arranged to provide active load stimulation to the bone-implant interface.

2. The bone implant of claim 1, wherein one or more of the actuatable elements are freely mobile within the implant, implant body, implant head and/or a removable implant head extension such that on experiencing movement, the elements translate, for example, longitudinally, transversally, and/or radially within the implant body, implant head and/or a removable implant head extension.

3. The bone implant of claim 1, wherein one or more actuatable elements comprise one or more discrete bodies that are freely mobile within the implant body, the implant head and/or the implant head extension.

4. The bone implant of claim 1, wherein one or more actuatable elements comprise grains, hollow or solid spheres, rolling or ball bearings, particles, and/or one or more translatable rods, pins, cylinders, or combinations thereof or a cannulated rod or cylinder.

5. The bone implant of claim 1, further comprising at least one chamber, region, passageway, cannulation or orifice in the implant body, implant head and/or the implant head extension, comprising the mechanical means, and optionally wherein the mechanical means is removable from the implant.

6. The bone implant of claim 1, wherein the bone implant is dissolvable in situ.

7. The bone implant of claim 1, wherein attachment of the implant head to the implant body is a moveable attachment wherein the implant head is translatable along a portion of a longitudinal axis of the implant and/or is rockable when positioned on the implant body, and optionally, the implant head is removeable from the implant body.

8. The bone implant of claim 1, wherein the mechanical means moves with the bone implant on kinetic movement of a subject having the implant, or the mechanical means move independently of subject's kinetic movement, and optionally, is controlled by a motor or other controlling device.

9. The bone implant of claim 1, wherein the implant body, implant head and/or the implant extension head is cannulated.

10. The bone implant of claim 1, comprising an implant body for insertion and retention of the implant into bone, an implant head engagable with a tool for insertion of the implant body into the bone, and optionally a neck portion for connecting the implant body with an implant head.

11. The bone implant of claim 1, further comprising an implant body cover and/or an implant head cover, and/or an implant extension cover for closing off internal parts from tissue ingress.

12. The bone implant of claim 11, wherein the cover is a temporary cover, and optionally is a pierceable cover formed from a layer of biocompatible material which can be penetrated.

13. The bone implant of claim 12, wherein the implant head extension and/or the temporary cover has a shape and/or configuration that enables location under the subject's skin, and optionally comprises a pointed or textured surface for tactile location under the skin or a radio opaque material for location via CT or radiological method.

14. The bone implant of claim 1 which is a pedicle screw and further comprises an implant set or locking screw or cap for locking a spinal rod into the implant head.

15. The bone implant of claim 14, wherein the locking screw or cap comprising the mechanical means, preferably wherein the mechanical means is removable from the locking screw or cap.

16. The bone implant of claim 1 used in the treatment and/or prevention of a bone wasting/weakening disease or disorder.

17. A surgical method for the treatment, prevention and/or correction of one or more of spinal stenosis, spondylolisthesis, spinal deformities, fracture, pseudoarthosis, tumour resection, failed previous fusion, degenerative disc disease, dislocation, scoliosis, and kyphosis, wherein the surgical method comprises the use of the bone implant of claim 1.

18. A kit of parts comprising a plurality of bone implants according to claim 1.

19. The kit of claim 18, further comprising a sterilisable housing which accommodates the plurality of the implants.

20. The kit of claim 18 which is a surgical instrumentation kit used in a spinal fixation surgical procedure.

21. The bone implant of claim 1, which when implanted promotes bone stimulation including bone growth, strengthening, densification and/or osseointegration.

22. The surgical method of claim 17 which is a two stage surgical method which includes:
a first stage surgical step whereby the bone implant of claim 1 is inserted into a bone at a first timepoint, and subsequently,
a second stage surgical step at a second timepoint in which a load is applied to the bone implant inserted in the first stage surgical step,
whereby the interval between the first step and the second step is sufficient to allow an acceptable level of osseointegration to occur between the implant and bone.
